(19) United States
(12) Patent Application Publication
(10) Pub. No.: US 2003/0049250 A1
(43) Pub. Date: Mar. 13, 2003

(54) Method for enhancing endothelial function in humans

(76) Inventors: Matti Karvonen, Turku (FI); Markku Koulu, Turku (FI); Ullamari Pesonen, Turku (FI); Tapani Ronnemaa, Piispanristi (FI); Mikko Jarvisalo, Turku (FI); Laura Jartti, Turku (FI); Olli Raitakari, Turku (FI)

Correspondence Address: ROTHWELL, FIGG, ERNST & MANBECK, P.C., 1425 K STREET, N.W., SUITE 800, WASHINGTON, DC, 20005, US

(21) Appl. No.: 09/946,174

(22) Filed: Sep. 5, 2001

(51) Int. Cl. A61K 39/395; A61K 31/00

(52) U.S. Cl. 424/143.1; 514/1

(57) ABSTRACT

The present invention concerns a method for enhancing the endothelial function in humans, comprising administering to the person an NPY receptor active agent, wherein said receptor is present in the endothelial tissue. Furthermore, the invention concerns methods for the treatment or prevention of atherosclerotic vascular diseases; vascular spasm associated with angina pectoris; micro- or macrovascular complications of diabetes; premature ejaculation and impotence; or any disease or disorder where a deficit in the formation of nitric oxide for the vascular endothelium appears evident, said methods comprising administering to the person an NPY receptor active agent, wherein said receptor is present in the endothelial tissue.

### FIELD OF THE INVENTION

This invention relates to a method for enhancing the endothelial function in humans and to methods for the treatment or prevention of diseases and disorders of endothelial origin and due to insufficient endothelial function.

### BACKGROUND OF THE INVENTION

The publications and other materials used herein to illuminate the background of the invention, and in particular, cases to provide additional details respecting the practice, are incorporated by reference and listed in the Bibliography.

NPY is a neurotransmitter of the sympathetic nervous system, co-stored with noradrenaline in peripheral sympathetic nerve endings and released in response to strenuous sympathetic stimulation (Lundberg, Terenius, et al. 1982 (1)). When released from peripheral nerve terminals to arterial periadventitia NPY causes direct endothelium-independent vasoconstriction via stimulation vascular smooth-muscle cell receptors (Edvinsson, Emson, et al. 1983 (2); Edvinsson 1985 (3); Abounader, Villemure, et al. 1995 (4)).

Although intraluminal NPY infusion can cause coronary vasoconstriction in patients with coronary heart disease (CHD) (Clarke, Davies, et al. 1987 (5)), the activation of endothelial NPY receptors leads to endothelium-dependent, NO-mediated vasodilatation (Kobari, Fukuuchi, et al. 1993 (6); Torffvit & Edvinsson 1997 (7); You, Edvinsson, et al. 2001 (8)). Therefore NPY seems to have a dual role in the control of vascular tone.

NPY is widely expressed in the central and peripheral nervous systems and has many physiological functions such as in the control of metabolism and endocrine functions and in regulation of cardiovascular homeostasis. NPY induces endothelium-independent vasoconstriction (Budai, Vu, et al. 1989 (9); Gustafsson & Nilsson 1990 (10); of peripheral, cerebral and coronary arteries (Clarke, Davies, et al. 1987 (5)) by activating receptors on smooth muscle cells (You, Edvinsson, et al. 2001 (8)) and also potentiates the vasoconstriction induced by norepinephrine (Westfall, Yang, et al. 1995 (11); Fabi, Argiolas, et al. 1998 (12)). It has been estimated that NPY might account for approximately 30% of the vasoconstriction after sympathetic stimulation (Han, Chen, et al. 1998 (13)). NPY-induced vasoconstriction has been proposed to play a role in several cardiovascular diseases including acute myocardial infarction and angina pectoris (Ullman, Franco-Cereceda, et al. 1990 (14)), heart failure (Maisel, Scott, et al. 1989 (15); Hulting, Sollevi, et al. 1990 (16)) and hypertension (Chalmers, Morris, et al. 1989 (17); Solt, Brown, et al. 1990 (18); Lettgen, Wagner, et al. 1994 (19)). Most of the studies assessing NPY-induced vasoconstriction in humans or experimental animals in vivo have, however, involved application of exogenous NPY or NPY-agonists in large non-physiological doses and data on vasoconstrictory effects of endogenous NPY are ambiguous (Tanaka, Mori, et al. 1997 (20)).

In addition to release from peripheral nerve endings to arterial periadventitia, NPY and NPY mRNA are also expressed extraneuronally in the endothelium of peripheral vessels (Loesch, Maynard, et al. 1992 (21); Zukowska-Grojec, Karwatowska-Prokopczuk, et al. 1998 (22)). The minor proportion of circulating NPY level, derived from the endothelial cells has been implicated to act as an autocrine and paracrine mediator and to stimulate its receptors Y1 and Y2 found on the endothelium (Sanabria & Silva 1994 (23); Jackerott & Larsson 1997 (24); Zukowska-Grojec, Karwatowska-Prokopczuk, et al. 1998 (22)). Recent studies have demonstrated that stimulation of endothelial NPY receptors leads to vasodilatation (Kobari, Fukuuchi, et al. 1993 (6); Torffvit & Edvinsson 1997 (7)) primarily through Y2 receptor activation (You, Edvinsson, et al. 2001 (8)). In addition to NPY, the endothelium can also produce NPY[3-36], a more specific Y2 agonist, from circulating native NPY by a serine protease dipeptidyl peptidase IV (Mentlein, Dahms, et al. 1993 (25)). You and coworkers (You, Zhang, et al. 1995 (26)) have shown that inhibition of NO-synthase or removal of the endothelial layer abolishes the dilatation response to NPY suggesting that the dilatation is NO-mediated (You, Edvinsson, et al. 2001 (8)). Therefore, the physiological role of NPY in the control of arterial tone seems to be of dual nature causing vasoconstriction via activation of smooth-muscle receptors and on the other hand increased NO-release and vasodilatation via endothelial receptor activation (You, Edvinsson, et al. 2001 (8)). The dilator response to physiological NPY-concentrations is rather weak, and only 40% of the dilatation produced by intraluminal application of ATP, a potent endothelium-dependent vasodilatator (You, Edvinsson, et al. 2001 (8)). Therefore, it has been suggested that the role of NPY receptors in the endothelium is mainly to potentiate dilatation produced by more powerful vasodilatators in a similar manner as it does with constrictor agents that stimulate smooth muscle receptors (You, Edvinsson, et al. 2001 (8)).

It was recently reported that a rather common Leu7Pro polymorphism located in the signal peptide of the prepro-NPY is associated with increased intracellular processing and release of vascular endothelial NPY in carriers of the Pro7 substitution (Kallio, Pesonen, et al. 2001 (27)). The present study was undertaken to assess the impact of Leu7Pro polymorphism on arterial endothelial function. Brachial artery flow-mediated dilatation (FMD) was measured using high-resolution ultrasound in two independent and dissimilar populations to obtain reliable results. Study population I included all 152 middle-aged men participating in a study of cardiovascular risk factors and early morphological and functional atherosclerotic changes. Study population II consisted of 95 children from a large coronary risk factor intervention study.

### SUMMARY OF THE INVENTION

Thus, according on one aspect, this invention concerns a method for enhancing the endothelial function in humans, comprising administering to the person an NPY receptor active agent, wherein said receptor is present in the endothelial tissue.

According to further aspects, the invention concerns methods for the treatment or prevention of

atherosclerotic vascular diseases;

vascular spasm associated with angina pectoris;

micro- or macrovascular complications of diabetes;

premature ejaculation and impotence; or

any disease or disorder where a deficit in the formation of nitric oxide for the vascular endothelium appears evident,

said methods comprising administering to the person an NPY receptor active agent, wherein said receptor is present in the endothelial tissue.

### BRIEF DESCRIPTION OF THE DRAWINGS

FIG. 1 shows flow-mediated dilatation values of the brachial artery in the adult subjects with Leu7/Leu7 and Pro7/−NPY signal peptide genotype. The horizontal lines present the 5^{th}, 25^{th}, 50^{th} (the median), 75^{th} and 95^{th} centiles. The broken horizontal lines present the means. Significance for the difference between the means, p=0.045.

FIG. 2 shows temporal development of brachial artery flow-mediated dilatation response in children with Leu7/Leu7 ( ) and Pro7/−( ) NPY signal peptide genotype. Significance for the difference in area under the FMD-time-curve, p=0.03. Mean and SEM are shown.

### DETAILED DESCRIPTION OF THE INVENTION

The term “enhancing endothelial function” means by acting on neuropeptide Y receptors located in vascular endothelial cells to improve and/or restore deteriorated endothelial function. It includes, for example, increased vasodilatation after stimulation of endothelial cells to produce endothelium derived relaxin factor, nitric oxide, but it is not restricted hereto. This term “enhancing endothelial function” includes further prevention of predisposition to thrombosis, leucocyte adhesion, inflammation and proliferation of smooth muscle cells in the arterial wall, which are consequences of deteriorating endothelial function. Enhancing endothelial function may result in slowing down of the development of atherosclerosis. Generally, this term “enhancing endothelial function” could be defined as improving of deteriorated endothelial function resulting from hypertension, hyperlipidemia, diabetes, infection, hereditary factors, smoking and other factors.

Diseases or disorders that can be treated or prevented by enhancing the endothelial function in a person, are for example atherosclerotic vascular diseases such as coronary heart disease, peripheral atherosclerosis, cerebral atherosclerosis and vascular dementia; vascular spasm associated with angina pectoris; micro- and macrovascular complications of diabetes such as coronary heart disease, diabetic retinopathy, diabetic nephropathy and diabetic erectile dysfunction; premature ejaculation and impotence; and in all other diseases or disorders, including erectile dysfunction and premature ejaculation, where a deficit in the formation of nitric oxide for the vascular endothelium appears evident.

Impairment of vascular endothelial cell function or endothelial dysfunction is an early physiological event in the pathogenesis in atherosclerosis (Healy B. 1990 (28)). Endothelial dysfunction occurs in vitro in the earliest stages, before plaques exist and certainly before clinical manifestations and detection of disease. Endothelial dysfunction predisposes thrombosis, leucocyte adhesion, and proliferation of artery wall smooth muscle cells (Ross 1986 (29)). In arteries with healthy endothelium, increased blood flow causes dilatation of the vessel (Laurent et al. 1990 (30); Rubanyi et al. 1986 (31)), via release of endothelium-derived relaxing factor (Pohl et al. 1986 (32)), whereas this release and subsequent dilatation falls in endothelial dysfunction (Wendelhag et al. 1991 (33)).

The normalizing effect on the endothelial function can also offer therapeutic opportunities in several diseases of vascular origin, such as atherosclerosis, in any vascular spasm processes, in complications of diabetes (Durante et al. 1988 (34)) and in all disorders, including premature ejaculation (Hull et al. 1994 (35)), where a deficit in the formation of nitric oxide for the vascular endothelium appears evident.

In particular, it has been demonstrated that the erection of the penis is modulated by nitric oxide produced in the endothelium (Burnett et al. 1992 (36); Rajfer et al., 1992 (37)) and that, under circumstances where the endothelial function is detrimentally affected, as in hyperlipidemias (Kugiyama et al. 1990 (38)), correction of the detrimental change normalizes the erectile function, measured with accuracy during nocturnal sleep (Kostis et al. 1994 (39); Rosen 1995 (40)). The normalization of the impaired endothelial function obtained with a neuropeptide Y receptor agonist acting on neuropeptide Y receptors located in endothelial cells can represent an entirely novel therapeutic approach to treat atherosclerosis, any vascular spasm processes, vascular complications of diabetes, premature ejaculation and impotence in patients with vascular disorders with various causes and due to endothelial dysfunction (diabetes, arteriosclerosis, and the like) and in patients where only a functional disorder can be detected.

The term “NPY receptor” shall be understood to mean a receptor active for NPY or a peptide fragment of NPY. Such a fragment can, for example, be the peptide fragment of [D-Arg(25)]-NPY (Mullins et al. 2001 (41)), [Leu³¹, Pro³⁴]NPY (Potter et al. 1992 (42)), NPY₃₋₃₆, NPY₁₃₋₃₆ (Wimalawansa 1995 (43), Grandt el al. 1996 (44)) or the like.

The term “agent” shall be understood to include the compound itself (racemic form as well as isomers) and any pharmaceutically acceptable derivatives thereof, such as salts or esters.

Said NPY receptor shall be a NPY binding receptor that is present in the endothelial tissue. As examples of such receptors can be mentioned the Y1 and Y2 receptors (Zukowska-Grojec et al. 1998 (22)).

The active agent to be administered can in principle be either an agonist or an antagonist, or a combination of an agonist in a said endothelial receptor and an antagonist in another receptor. The same agent can thus be an agonist in said endothelial receptor and an antagonist in another receptor. Alternatively, a mixture of an agonist in said endothelial receptor and an antagonist in another receptor can also be administered.

According to a preferable embodiment of this invention, the agent is an NPY receptor agonist, preferably a Y2 or Y1 receptor agonist, most preferably a Y2 receptor agonist. Y2 receptor agonists have been described before in the literature. As examples can be mentioned NPY₃₋₃₆ and [Leu³¹, Pro³⁴]NPY. The suitable agent is, however, not restricted to the aforementioned examples. Any compound acting as a Y2 receptor agonist is useful in the method according to this invention.

It is also believed that a combination of action on the Y2 and Y1 receptor would be useful.

Through stimulation of Y1 receptors NPY stimulates smooth muscle cell DNA synthesis (Zukowska-Grojec et al. 1993 (45); Nilsson et al. 2000 (46). NPY also causes vasocinstriction via Y1 receptors in smooth muscle (Xia et al. 1992 (47); Nilsson et al. 1996 (48); Franco-Cereceda and Liska 1998 (49)). However, Y1 stimulation may also cause vasodilatation trough increase in nitric oxide production in endothelial cells (Nilsson et al. 2000 (46)). Therefore, stimulating endothelial cell Y1 receptors, but not smooth muscle cell Y1 receptors, may also have endothelial function enhancing effect.

An antagonistic molecule with a property of intrinsic NPY receptor stimulating activity on Y2 and or Y1 receptors, which by acting on endothelial NPY Y2 and/or Y1 receptors enhances endothelial function, and which by acting on other than endothelial NPY Y2 and/or Y1 receptors blocks vasoconstrictive and smooth muscle cell proliferative actions (potential atherosclerotic promoting effects of excess endogenous NPY) of endogenous NPY.

Thus, according to another embodiment of this invention the Y2 receptor agonist is also a Y1-receptor agonist or antagonist.

According to a further embodiment, a separate Y1 receptor agonist or antagonist is administered in combination with the Y2 receptor agonist.

For the purpose of this invention, the NPY receptor active agent can be administered by various routes. The suitable administration forms include, for example, oral formulations; parenteral injections including intravenous, intramuscular, intradermal and subcutaneous injections; and transdermal, intraurethral or rectal formulations; and inhaled formulations. Suitable oral formulations include e.g. conventional or slow-release tablets and gelatine capsules.

The required dosage of the NPY receptor active compounds will vary with the particular condition being treated, the severity of the condition, the duration of the treatment, the administration route and the specific compound being employed.

The invention will be illuminated by the following non-restrictive Examples. Standard techniques well known in the art or the techniques specifically described below are utilized.

### EXAMPLE 1

### Subjects And Methods

A. Subjects

Middle-aged men: The study population consisted of 152 male twin subjects discordant for permanent migration from Finland to Sweden identified from the Finnish Twin Cohort Study (Kaprio, Sarna, et al. 1978 (50)). Their mean age was 54±7 (range 42-69) years. The characteristics of study subjects, use of medication, and previously diagnosed cardiovascular diseases are shown in Table 1. Twenty men had Pro7 substitution in prepro-NPY. All subjects gave their informed consent.

Healthy children: The subjects were enrolled from the Special Turku Coronary Risk Factor Intervention Project (STRIP), which is an ongoing investigation aimed to study the effects of dietary intervention on coronary risk factors in young children (Niinikoski, Viikari, et al. 1996 (51)). We studied 95 (21 with Pro7 substitution) 9 to 11-year old children who were prospectively chosen from the main study population (n=1054) according to previously performed prepro-NPY genotype determinations. The objective was to study ca. 100 children, >20 of which, carrying the Pro7 substitution. The wild type subjects were selected randomly to the study from the main population. The frequency of Pro7 substitution in the main study population is 12.4% (Karvonen, Koulu, et al. 2000 (52)). None of the children included in the study were homozygous for the Pro7 mutation. The different genotype groups were matched in terms of age, sex and body size. Written informed consent was acquired from the legal guardians of the children and they were also encouraged to get an approval from the child.

B. Study Protocol

Both studies were conducted according to the guidelines of the Helsinki declaration and the study protocols had been approved by the Joint Ethics Committee of Turku University and Turku University Central Hospital.

Middle-aged men: All subjects were examined in the same laboratory and both cotwins in a pair on the same day. In the morning, the supine blood pressure was measured after 5 minutes rest using a standard mercury sphygmomanometer. Hypertension was defined as the use of antihypertensive medication and/or actual blood pressure ≧160/95 mmHg. Body fat was assessed using bioelectric impedance analysator (BIA-101 A/S, RJL systems Inc., Clemens, Mich.). Thereafter, a standard 75 g oral glucose tolerance test was performed. Diabetes was defined as the use of antidiabetic medication and/or fasting serum glucose ≧7.0 mmol/L and/or 2-h glucose ≧11.1 mmol/L.

In the afternoon, after a light lunch (approximately 450 kcal) the subjects underwent the assessment of endothelial function and stress echocardiography. All ultrasound scans were performed by a single experienced vascular sonographer and the images were analyzed by two independent observers.

Healthy children: All ultrasound studies were performed by a single experienced vascular sonographer in the morning between 7.30 and 9.00 AM after a 10 hour overnight fast and after drawing of venous blood samples. Studies were carried out in a clinical research laboratory and all procedures were undertaken in silence to minimize external stimuli. Blood pressure was measured 3 times during the examination using a standard pneumatic sphygmomanometer with the subject in supine position.

C. Endothelial Function

All studies were performed using an Acuson Sequoia 512 mainframe (Acuson, Mountain View, Calif.) with a 13.0 MHz linear array transducer. Brachial artery diameter was measured from B-mode ultrasound images. In all studies, scans were obtained at rest and during reactive hyperemia. The subjects laid quietly for 10 minutes before the first scan. The left brachial artery was scanned in longitudinal section approximately 2-15 cm above the antecubital crease. Depth and gain settings were set to optimize images of the lumen/arterial wall interface, images were magnified using a resolution box function and the operating parameters were not changed during the study. When a satisfactory transducer position was found, the position was marked on the skin and the arm remained in the same position throughout the study. A resting scan was performed and arterial flow velocity was measured using a Doppler signal. Increased flow was then induced by inflation of a pneumatic tourniquet placed around the forearm (distal to the scanned part of the artery) to a pressure of 250 mmHg for 4.5 minutes, followed by release (Celermajer, Sorensen, et al. 1992 (53)).

A second scan was taken 45-75 seconds after the cuff deflation in middle-aged men and 40-180 seconds after cuff-release in children. The flow velocity recording was repeated during the first 15 seconds after the cuff was released. All brachial ultrasound scans were recorded on super-VHS videotapes for later analysis. Vessel diameter was always measured independently by two observers who were unaware of the subject's identity. The arterial diameter was measured at a fixed distance from an anatomic marker (e.g. a fascial plane) using ultrasonic calipers. Measurements were taken at end-diastole (incident with the R-wave on a continuously recorded ECG) from the anterior to the posterior intima-lumen interface (i-line) in middle-aged men and from the anterior to the posterior media-adventitia interface (m-line) in children to acquire reliable results (Jarvisalo, Jartti, et al. 2000 (54)).

In the adult subjects hyperemic diameter was measured at 60 sec after cuff-deflation. In children the hyperemic diameter was measured every 10 sec between 40-120 sec post-occlusion and every 15 sec between 120-180 sec post-occlusion (a total of 13 measurements) and the values were used to calculate the area under the FMD-time-curve (AUC). In middle-aged men the vessel was allowed to recover for 10-15 minutes, after which a resting scan was taken. Then a sublingual glyceryl trinitrate (GTN) spray (400 μg) was given and after 4 minutes the vessel diameter was measured in order to determine the endothelium independent vasodilation.

As for the children, no GTN was administered to avoid possible discomfort. The vessel diameter in scans after reactive hyperemia and nitroglycerin administration was expressed as the percentage relative to the resting scan (100 percent). This method has been previously shown by us (Jarvisalo, Jartti, et al. 2000 (54)) and others (Sorensen, Celermajer, et al. 1995 (55)) to be accurate and reproducible for measurement of small changes in arterial diameter.

In the present study, the between observer reliability was estimated by calculating intraclass correlation coefficients between the two observers. There was good agreement between the observers both in the measurement of the brachial artery diameter in baseline and FMD percent, the intraclass correlations being 0.998 and 0.964, respectively (Jarvisalo, Jartti, et al. 2000 (54)). Mean values of the two analyzers were used in statistical analyses.

D. Biochemical Methods

Blood samples were drawn after an overnight fast from an antecubital vein. Serum glucose was measured by the glucose dehydrogenase method (Merck Diagnostica, Darmstadt, Germany). Cholesterol and triglyceride concentrations were determined enzymatically (Merck, Darmstadt, Germany) in an autoanalyzer (AU 510; Olympus, Hamburg, Germany). For the analysis of HDL and LDL cholesterol, serum was centrifuged (18 h, 105,000×g) at a density of 1.006 g/ml to separate the very-low-density lipoprotein (VLDL) fraction. After removing VLDL, LDL was precipitated from the infranatant (HDL+LDL) with dextrane sulphate 500,000-magnesium chloride, and cholesterol content of the infranatant and supernatant (HDL cholesterol) were determined enzymatically according to the method of Kostner (Kostner 1976 (56)). In the children low-density lipoprotein cholesterol (LDL-C) concentration was calculated using Friedewald's equation (Friedewald, Levy, et al. 1972 (57)). None of the children had serum triglyceride levels over 2 mmol/l.

DNA samples were isolated from dried whole blood collected on filter paper (children) or from EDTA-whole blood specimens (men). DNA was extracted using a DNA isolation kit (Gentra Systems, Minneapolis, Minn.) as suggested by the manufacturer. The prepro-NPY genotype was determined by PCR-restriction fragment length polymorphism analysis from extracted DNA by an investigator unaware of the subjects' clinical details. Thymidine 1128 to cytosine 1128 substitution generates a BsiEI restriction site, which was used to genotype the subjects for the Leu7Pro polymorphism (Karvonen, Koulu, et al. 2000 (52)).

E. Stress Echocardiography

All adult subjects underwent maximal bicycle exercise testing using a protocol with 20 W load increments every minute. Subjects exercised until limited by fatigue. Three leads of electrocardiogram were continuously recorded and a 12-lead electrocardiogram was obtained every minute of exercise. Blood pressure was measured at every load increment stage. Transthoracic echocardiography was performed using an Acuson Sequoia mainframe and 3.5 MHz transducer before the stress test to obtain baseline images and immediately after the stress test to obtain poststress images. For interpretation of stress echo images, we used the 16-segment model adopted by the American Society of Echocardiography (Armstrong, Pellikka, et al. 1998 (58)). Myocardial segments were considered ischemic if a new wall motion abnormality or worsening of pre-existing abnormalities was detected after the bicycle stress test. There were no ST-segment depressions without ischaemic finding in echo or a previous history for CHD. History of CHD was considered positive if a subject had a history of myocardial infarction or if a subject reported that he had been previously diagnosed with CHD and this was confirmed in the present examination. The adult subjects were grouped as having CHD if they had either a history of CHD or a ischaemic finding on stress echocardiography.

F. Statistical Methods

The results are expressed as mean±SD, unless stated otherwise. In the children, comparisons between the groups were conducted by student's t-test or wilcoxon's rank sum test, as appropriate. Univariate associations between the study variables were analyzed by calculating the Pearson's correlation coefficients. Multivariate analyses were done using linear regression technique.

While the adult study population consists of twins from twin pairs, the analysis considered the data as individuals with differing genotypes. Statistical methods in general assume that subjects are independent observations, with uncorrelated error terms. Because the twins were sampled as twin pairs, and the error terms within pairs may be correlated, we used statistical techniques to take this into account and derive correct standard errors and confidence intervals. Such statistical methods are used in studies with repeated measures or clustered data (e.g. family members sampled in households, pups in litters). Thus, the equality of means between genotype groups was tested using regression models with generalized estimating equations (GEE) (Zeger & Liang 1986 (59)) that specified that the individual observations were derived from twin pairs. To test the equality of distributions of dichotomous traits, a corresponding logistic regression model with GEE was used. To test whether the association between NPY genotype and FMD was independent of the effects of other covariates, these covariates were entered stepwise into a regression model with GEE. All data analysis was carried out in SAS (version 6.12, SAS Institute, Cary, N.C., USA), the GEE regression analyses in SAS PROC GENMOD.

### EXAMPLE 2

### Results and Analysis of Studies

Middle-aged men: Twenty of the adult subjects were carriers of the Pro7 substitution (13.2%) whereas 132 subjects had the Leu7Leu genotype. None of the subjects were homozygous for the Pro7 substitution. Of those with the Pro7 substitution eight men were monozygous twins (=4 pairs) and 12 were dizygous, 10 lived in Finland and 10 lived in Sweden. There were no significant differences between the groups in age, serum lipid levels, fasting glucose, 2-hour glucose, fat percentage, smoking habits, blood pressure, prevalence of hypertension or prevalence of diabetes but BMI was higher in subjects with the Pro7 substitution (Table 1). Only 22 subjects (14.5%) had BMI>30 kg/m² and none of them were carriers of the Pro7 substitution. Subjects with the substitution tended to have more coronary heart disease (CHD) (20% vs. 10%, p=0.17).

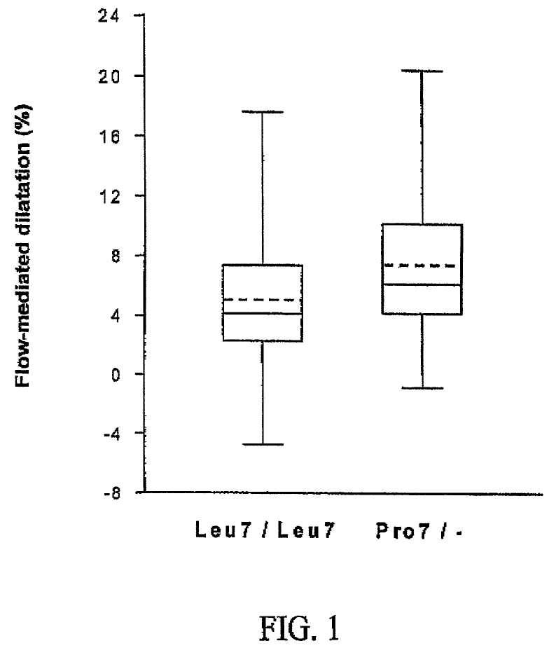

Brachial artery FMD was successfully assessed in 150 subjects. Two recordings had to be disqualified because of poor image quality. Brachial artery baseline diameter was similar between the genotypes (4.03±0.50 vs. 4.02±0.38 mm, p=0.87). Subjects with Pro7 substitution had 48% higher FMD60 compared to wild type subjects (Table 1, FIG. 1). The difference in FMD60 remained significant in a subanalysis (9.07±4.71% vs. 6.01±3.89%, p=0.015) including only subjects without any cardiovascular medication (n=94, 14 subjects with the polymorphism and 80 with the wild type). Furthermore the difference remained significant when all subjects with CHD were excluded (n=133, 16 subjects with the polymorphism and 117 with the wild type, 8.10±5.18 vs. 5.22±4.09%, p=0.028).

In a stepwise regression model, adjustment for age, country of residence, zygosity, BMI, basal vessel diameter, serum lipids, blood pressure, smoking, medication, diabetes, history for CHD and hypertension, did not abolish the difference in FMD between the two NPY signal peptide genotype groups (Table 2).

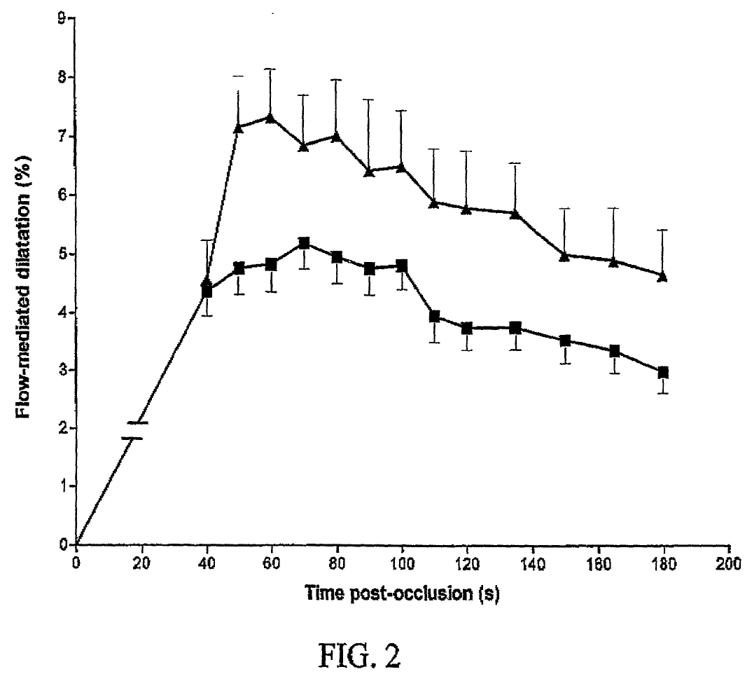

Children: The characteristics of the study groups are shown in Table 3. There were no significant differences in age, gender, serum lipids, blood pressure or body size between children with or without Pro7 substitution. Brachial artery baseline diameter was similar between the genotypes (3.0±0.3 vs. 3.1±0.3 mm, p=0.51). FMD responses of the study groups are depicted in FIG. 2. Children with Pro7 substitution had 52% higher FMD60 and 42% higher AUC compared to wild type children (Table 3). The temporal development of the dilatation response was similar in the study groups (repeated measures analysis of variance, p=0.30). In the multivariate regression model including age, gender, body mass index, blood pressure, LDL-C, HDL-C and brachial baseline diameter as explanatory variables, the prepro-NPY genotype explained significantly (β=2.55, p=0.018) the variation in FMD60.

TABLE 1
Background and ultrasound data of the middle-aged men
with or without Pro7 substitution in the prepro NPY.
Values are mean ± SD or prevalences (%).

| | Wild type | Pro7 substitution | p-value |
|---|---|---|---|
| Number of subjects | 132 | 20 | — |
| Age (years) | 53.7 ± 7.0 | 56.6 ± 6.4 | 0.16 |
| BMI (kg/m²) | 26.7 ± 4.4 | 25.0 ± 2.2 | 0.022 |
| Fat percentage (%) | 22.9 ± 5.7 | 22.0 ± 4.7 | 0.47 |
| Systolic blood pressure | 137.5 ± 21.5 | 133.3 ± 15.1 | 0.35 |
| (mmHg) | | | |
| Diastolic blood pressure | 83.9 ± 11.4 | 82.2 ± 8.5 | 0.43 |
| (mmHg) | | | |
| Hypertension (%) | 50/132 (38%) | 4/20 (20%) | 0.12 |
| Total cholesterol (mmo/L) | 5.79 ± 1.13 | 5.56 ± 0.73 | 0.28 |
| HDL-cholesterol (mmol/L) | 1.48 ± 0.44 | 1.39 ± 0.32 | 0.32 |
| LDL-cholesterol (mmol/L) | 3.60 ± 0.96 | 3.53 ± 0.63 | 0.71 |
| Triglycerides (mmol/L)* | 1.60 ± 0.98 | 1.44 ± 0.57 | 0.78 |
| Fasting glucose (mmol/L) | 6.05 ± 1.58 | 5.67 ± 0.93 | 0.21 |
| Diabetes (%) | 19/132 (14%) | 3/20 (15%) | 0.96 |
| CHD | 13/132 (10%) | 4/20 (20%) | 0.17 |
| Smoking | | | 0.29 |
| Non-smoker (%) | 33/132 (25%) | 5/20 (25%) | |
| ex-smoker (%) | 46/132 (35%) | 10/20 (50%) | |
| occasional smoker (%) | 6/132 (5%) | 2/20 (10%) | |
| smoker (%) | 47/132 (36%) | 3/20 (15%) | |
| Any medication (%) | 51/132 (39%) | 6/20 (30%) | 0.52 |
| Cholesterol lowering | 10/132 (8%) | 3/20 (15%) | 0.30 |
| agent (%) | | | |
| Brachial artery | 4.03 ± 0.50 | 4.02 ± 0.38 | 0.87 |
| diameter (mm) | | | |
| Flow-mediated dilatation at | 4.99 ± 4.03 | 7.38 ± 4.95 | 0.045 |
| 60 sec post-occlusion (%) | | | |
| Glyceryl trinitrate mediated | 12.9 ± 6.60 | 15.2 ± 6.76 | 0.12 |
| dilatation (%) | | | |

*statistical analysis after logarithmic transformation

TABLE 2
Association between prepro NPY genotype and
flow-mediated vasodilation in
multivariate analysis in adult subjects.

| | Model | β ± SE | p-value |
|---|---|---|---|
| A. | NPY | −2.39 ± 1.19 | 0.045 |
| B. | A + age, BMI, country, vessel size, | −2.80 ± 1.09 | 0.010 |
| | zygosity | | |
| C. | B + total, HDL-, LDL-cholesterol, | −2.77 ± 1.10 | 0.012 |
| | triglycerides | | |
| D. | C + systolic and diastolic BP | −2.69 ± 1.15 | 0.019 |
| E. | D + smoking | −2.52 ± 1.06 | 0.018 |
| F. | E + medication | −2.28 ± 0.93 | 0.014 |
| G. | F + diabetes | −2.29 ± 0.93 | 0.014 |
| I. | G + CHD | −2.42 ± 0.95 | 0.011 |
| J. | H + hypertension | −2.42 ± 0.96 | 0.011 |

TABLE 3
Background and ultrasound data of the school-aged
children with or without Pro7 substitution in the prepro NPY.
Values are mean ± SD.

| | | Pro7 | |
|---|---|---|---|
| | Wild type | substitution | p-value |
| Number of subjects (boys) | 74 (40) | 21 (10) | — |
| Age (years) | 10 ± 0.6 | 10 ± 0.6 | 0.10 |
| BMI (kg/m²) | 17.6 ± 3.2 | 18.1 ± 2.4 | 0.55 |
| Ponderal index (kg/m³) | 12.3 ± 2.0 | 12.5 ± 1.6 | 0.59 |
| Systolic blood pressure (mmHg) | 107 ± 8 | 105 ± 6 | 0.44 |
| Diastolic blood pressure (mmHg) | 63 ± 5 | 62 ± 4 | 0.51 |
| Total cholesterol (mmol/L) | 4.4 ± 0.8 | 4.2 ± 0.9 | 0.45 |
| HDL-cholesterol (mmol/L) | 1.46 ± 0.35 | 1.45 ± 0.38 | 0.93 |
| LDL-cholesterol (mmol/L) | 2.6 ± 0.7 | 2.5 ± 0.7 | 0.48 |
| Triglycerides (mmol/L)* | 0.65 ± 0.27 | 0.71 ± 0.31 | 0.39 |
| Brachial artery diameter (mm) | 3.0 ± 0.3 | 3.1 ± 0.3 | 0.69 |
| Flow-mediated dilatation at | 4.8 ± 4.0 | 7.3 ± 3.7 | 0.013 |
| 60 sec post-occlusion (%) | | | |
| Area under the FMD-time- | 549 (489) | 782 (692) | 0.031 |
| curve (% sec) | | | |

This study demonstrates that subjects with Pro7 substitution in the signal peptide region of the prepro-NPY show enhanced systemic endothelial function, measured as flow-mediated arterial dilatation, compared to wild type subjects. Furthermore, the difference in endothelial function between the genotypes was observed in two totally independent and dissimilar study populations; one consisting of middle-aged men, the other of school-aged children.

Endothelial dysfunction is an early event in atherogenesis (Ross 1993 (60)), already present in children with coronary risk factors (Celermajer, Sorensen, et al. 1992 (53); Mietus-Snyder & Malloy 1998 (61)). The brachial artery dilator response to increased shear stress has been shown to be due mainly to endothelial release of nitric oxide (Joannides, Haefeli, et al. 1995 (62)), and to correlate with invasive testing of coronary endothelial function (Anderson, Uehata, et al. 1995 (63)), as well as with the extent and severity of coronary atherosclerosis (Neunteufl, Katzenschlager, et al. 1997 (64)). By the use of this method endothelial dysfunction has been shown to associate with several traditional coronary risk factors, dyslipidemia, diabetes and smoking (Celermajer, Sorensen, et al. 1993 (65); Raitakari, Adams, et al. 1999 (66)).

Our finding of enhanced flow-mediated dilatation in carriers of Pro7 substitution is surprising as previous studies have linked the substitution with increased, not decreased risk of atherosclerosis in terms of high serum cholesterol in obese adults and slightly increased intima-media thickness in type 2 diabetics (Karvonen, Pesonen, et al. 1998 (67); Niskanen, Rauramaa, et al. 1996 (68)). There is, however, insufficient knowledge on the cellular mechanisms responsible for both pro- and antiatherogenic effects of NPY, to make conclusions of the significance of this polymorphism in the development of subclinical atherosclerosis. As the difference in FMD between the prepro-NPY genotypes was also seen in children, in addition to adult subjects, it seems therefore, plausible that the association between prepro-NPY polymorphism and endothelial function is of physiological nature, not caused by effects of coronary risk factors and thereby independent of the possibly increased risk for CHD in the carriers of Pro7.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### Bibliography

1. Lundberg J M, Terenius L, Hokfelt T et al. Neuropeptide Y (NPY)-like immunoreactivity in peripheral noradrenergic neurons and effects of NPY on sympathetic function. Acta Physiol Scand 1982;116:477-80.

2. Edvinsson L, Emson P, McCulloch J, Tatemoto K, Uddman R. Neuropeptide Y: cerebrovascular innervation and vasomotor effects in the cat. Neurosci Lett 1983;43: 79-84.

3. Edvinsson L. Characterization of the contractile effect of neuropeptide Y in feline cerebral arteries. Acta Physiol Scand 1985; 125:33-41.

4. Abounader R, Villemure J G, Hamel E. Characterization of neuropeptide Y (NPY) receptors in human cerebral arteries with selective agonists and the new Y1 antagonist BIBP 3226. Br J Pharmacol 1995; 116:2245-50.

5. Clarke J G, Davies G J, Kerwin R et al. Coronary artery infusion of neuropeptide Y in patients with angina pectoris. Lancet 1987;1:1057-9.

6. Kobari M, Fukuuchi Y, Tomita M et al. Transient cerebral vasodilatory effect of neuropeptide Y mediated by nitric oxide. Brain Res Bull 1993;31:443-8.

7. Torffvit O, Edvinsson L. Blockade of nitric oxide decreases the renal vasodilatory effect of neuropeptide Y in the insulin-treated diabetic rat. Pflugers Arch 1997;434:445-50.

8. You J, Edvinsson L, Bryan R M, Jr. Neuropeptide Y-mediated constriction and dilation in rat middle cerebral arteries. J Cereb Blood Flow Metab 2001;21:77-84.

9. Budai D, Vu H Q, Duckles S P. Endothelium removal does not affect potentiation by neuropeptide Y in rabbit ear artery. Eur J Pharmacol 1989;168:97-100.

10. Gustafsson H, Nilsson H. Endothelium-independent potentiation by neuropeptide Y of vasoconstrictor responses in isolated arteries from rat and rabbit. Acta Physiol Scand 1990;138:503-7.

11. Westfall T C, Yang C L, Curfman-Falvey M. Neuropeptide-Y-ATP interactions at the vascular sympathetic neuroeffector junction. J Cardiovasc Pharmacol 1995;26:682-7.

12. Fabi F, Argiolas L, Ruvolo G, del Basso P. Neuropeptide Y-induced potentiation of noradrenergic vasoconstriction in the human saphenous vein: involvement of endothelium generated thromboxane. Br J Pharmacol 1998;124: 101-10.

13. Han S, Chen X, Cox B et al. Role of neuropeptide Y in cold stress-induced hypertension. Peptides 1998;19:351-8.

14. Ullman B, Franco-Cereceda A, Hulting J, Lundberg J M, Sollevi A. Elevation of plasma neuropeptide Y-like immunoreactivity and noradrenaline during myocardial ischaemia in man. J Intern Med 1990;228:583-9.

15. Maisel AS, Scott NA, Motulsky H J et al. Elevation of plasma neuropeptide Y levels in congestive heart failure. Am J Med 1989;86:43-8.

16. Hulting J, Sollevi A, Ullman B, Franco-Cereceda A, Lundberg J M. Plasma neuropeptide Y on admission to a coronary care unit: raised levels in patients with left heart failure. Cardiovasc Res 1990;24: 102-8.

17. Chalmers J, Morris M, Kapoor V et al. Neuropeptide Y in the sympathetic control of blood pressure in hypertensive subjects. Clin Exp Hypertens A 1989;11 Suppl 1:59-66.

18. Solt V B, Brown M R, Kennedy B, Kolterman O G, Ziegler M G. Elevated insulin, norepinephrine, and neuropeptide Y in hypertension. Am J Hypertens 1990;3:823-8.

19. Lettgen B, Wagner S, Hanze J, Lang R E, Rascher W. Elevated plasma concentration of neuropeptide Y in adolescents with primary hypertension. J Hum Hypertens 1994;8:345-9.

20. Tanaka E, Mori H, Chujo M et al. Coronary vasoconstrictive effects of neuropeptide Y and their modulation by the ATP-sensitive potassium channel in anesthetized dogs. J Am Coll Cardiol 1997;29:1380-9.

21. Loesch A, Maynard K I, Burnstock G. Calcitonin gene-related pep. Neuroscience 1992;48:723-6.

22. Zukowska-Grojec Z, Karwatowska-Prokopczuk E, Rose W et al. Neuropeptide Y: a novel angiogenic factor from the sympathetic nerves and endothelium. Circ Res 1998;83: 187-95.

23. Sanabria P, Silva W I. Specific 1251 neuropeptide Y binding to intact cultured bovine adrenal medulla capillary endothelial cells. Microcirculation 1994; 1:267-73.

24. Jackerott M, Larsson L I. Immunocytochemical localization of the NPY/PYY Y1 receptor in enteric neurons, endothelial cells, and endocrine-like cells of the rat intestinal tract. J Histochem Cytochem 1997;45:1643-50.

25. Mentlein R, Dahms P, Grandt D, Kruger R. Proteolytic processing of neuropeptide Y and peptide YY by dipeptidyl peptidase IV. Regul Pept 1993;49:133-44.

26. You J, Zhang W, Jansen-Olesen I, Edvinsson L. Relation between cyclic GMP generation and cerebrovascular reactivity: modulation by NPY and alpha-trinositol. Pharmacol Toxicol 1995;77:48-56.

27. Kallio J, Pesonen U, Kaipio K et al. Altered intracellular processing and release of neuropeptide Y due to leucine 7 to proline 7 polymorphism in the signal peptide of preproneuropeptide Y in humans. FASEB J 2001;15:1242-4.

28. Healy B. Endothelial cell dysfunction: an emerging endocrinopathy linked to coronary disease. J Am Coll Cardiol 1990;16:357-8.

29. Ross R. The pathogenesis of atherosclerosis—an update. N Engl J Med 1986;314:488-500.

30. Laurent S, Lacolley P, Brunel P, Laloux B, Pannier B, Safar M. Flow-dependent vasodilation of brachial artery in essential hypertension. Am J Physiol 1990;258:H1004-11.

31. Rubanyi G M, Romero J C, Vanhoutte P M. Flow-induced release of endothelium-derived relaxing factor. Am J Physiol 1986;250:Hl 145-9.

32. Pohl U, Holtz J, Busse R, Bassenge E. Crucial role of endothelium in the vasodilator response to increased flow in vivo. Hypertension 1986;8(1):37-44.

33. Wendelhag I, Gustavsson T, Suurkula M, Berglund G, Wikstrand J. Ultrasound measurement of wall thickness in the carotid artery: fundamental principles and description of a computerized analysing system. Clin Physiol 1991;11(6):565-77.

34. Durante W, Sen A K, Sunahara F A. Impairment of endothelium-dependent relaxation in aortae from spontaneously diabetic rats. Br J Pharmacol 1988;94(2):463-8.

35. Hull E M, Lumley L A, Matuszewich L, Dominguez J, Moses J, Lorrain D S. The roles of nitric oxide in sexual function of male rats. Neuropharmacology 1994;33(11):1499-504.

36. Burnett A L, Lowenstein C J, Bredt D S, Chang T S, Snyder S H. Nitric oxide: a physiologic mediator of penile erection. Science 1992;257(5068):401-3.

37. Rajfer J, Aronson W J, Bush P A, Dorey F J, Ignarro L J. Nitric oxide as a mediator of relaxation of the corpus cavemosum in response to nonadrenergic, noncholinergic neurotransmission. N Engl J Med 1992;326(2):90-4.

38. Kugiyama K, Kerns S A, Morrisett J D, Roberts R, Henry P D Impairment of endothelium-dependent arterial relaxation by lysolecithin in modified low-density lipoproteins. Nature 1990;344(6262):160-2.

39. Kostis J B, Rosen R C, Wilson A C. Central nervous system effects of HMG CoA reductase inhibitors: lovastatin and pravastatin on sleep and cognitive performance in patients with hypercholesterolemia. J Clin Pharmacol 1994;34(10):989-96.

40. Rosen R C: The Pharmacology of Sexual Function and Dysfunction, J. Bancroft (ed), Elsevier Sc., 1995, pp 277-287.

41. Mullins D, Kirby D, Hwa J, Guzzi M, Rivier J, Parker E Identification of potent and selective neuropeptide y y(1) receptor agonists with orexigenic activity in vivo. Mol Pharmacol 2001;60(3):534-40.

42. Potter E K, McCloskey M J. [Leu31, Pro34] NPY, a selective functional postjunctional agonist at neuropeptide-Y receptors in anaesthetised rats. Neurosci Lett 1992;134(2):183-6.

43. Wimalawansa S J. Purification and biochemical characterization of neuropeptide Y2 receptor. J Biol Chem 1995;270(31):18523-30.

44. Grandt D, Schimiczek M, Rascher W, Feth F, Shively J, Lee T D, Davis M T, Reeve J R Jr, Michel M C. Neuropeptide Y 3-36 is an endogenous ligand selective for Y2 receptors. Regul Pept 1996;67(1):33-7.

45. Zukowska-Grojec Z, Pruszczyk P, Colton C, Yao J, Shen G H, Myers A K, Wahlestedt C. Mitogenic effect of neuropeptide Y in rat vascular smooth muscle cells. Peptides 1993;14(2):263-8.

46. Nilsson T, Lind H, Brunkvall J, Edvinsson L. Vasodilation in human subcutaneous arteries induced by neuropeptide Y is mediated by neuropeptide Y Y1 receptors and is nitric oxide dependent. Can J Physiol Pharmacol 2000;78(3):251-5.

47. Xia J, Neild T O, Kotecha N. Effects of neuropeptide Y and agonists selective for neuropeptide Y receptor sub-types on arterioles of the guinea-pig small intestine and the rat brain. Br J Pharmacol 1992;107(3):771-6.

48. Nilsson T, Cantera L, Edvinsson L. Presence of neuropeptide Y Y1 receptor mediating vasoconstriction in human cerebral arteries. Neurosci Lett 1996;204(3): 145-8.

49. Franco-Cereceda A, Liska J. Neuropeptide Y Y1 receptors in vascular pharmacology. Eur J Pharmacol 1998;349(1):1-14.

50. Kaprio J, Sama S, Koskenvuo M, Rantasalo I. The Finnish Twin Registry: formation and compilation, questionnaire study, zygosity determination procedures, and research program. Prog Clin Biol Res 1978;24 Pt B: 179-84.

51. Niinikoski H, Viikari J, Ronnemaa T et al. Prospective randomized trial of low-saturated-fat, low-cholesterol diet during the first 3 years of life. The STRIP baby project. Circulation 1996;94:1386-93.

52. Karvonen M K, Koulu M, Pesonen U et al. Leucine 7 to proline 7 polymorphism in the preproneuropeptide Y is associated with birth weight and serum triglyceride concentration in preschool aged children. J Clin Endocrinol Metab 2000;85:1455-60.

53. Celermajer D S, Sorensen K E, Gooch V M et al. Non-invasive detection of endothelial dysfunction in children and adults at risk of atherosclerosis. Lancet 1992;340:1111-5.

54. Jarvisalo M J, Jartti L, Toikka J O, Hartiala J J, Ronnemaa T, Raitakari O T. Noninvasive assessment of brachial artery endothelial function with digital ultrasound and 13-MHz scanning frequency: feasibility of measuring the true inner luminal diameter using the intima-lumen interface. Ultrasound Med Biol 2000;26: 1257-60.

55. Sorensen K E, Celermajer D S, Spiegelhalter D J et al. Non-invasive measurement of human endothelium dependent arterial responses: accuracy and reproducibility. Br Heart J 1995;74:247-53.

56. Kostner G M. Letter: Enzymatic determination of cholesterol in high-density lipoprotein fractions prepared by polyanion precipitation. Clin Chem 1976;22:695.

57. Friedewald W T, Levy R I, Fredrickson D S. Estimation of the concentration of low-density lipoprotein cholesterol in plasma, without use of the preparative ultracentrifuge. Clin Chem 1972;18:499-502.

58. Armstrong W F, Pellikka P A, Ryan T, Crouse L, Zoghbi W A. Stress echocardiography: recommendations for performance and interpretation of stress echocardiography. Stress Echocardiography Task Force of the Nomenclature and Standards Committee of the American Society of Echocardiography. J Am Soc Echocardiogr 1998;11:97-104.

59. Zeger S L, Liang K Y. Longitudinal data analysis for discrete and continuous outcomes. Biometrics 1986;42:121-30.

60. Ross R. The pathogenesis of atherosclerosis: a perspective for the 1990s. Nature 1993;362:801-9.

61. Mietus-Snyder M, Malloy M J. Endothelial dysfunction occurs in children with two genetic hyperlipidemias: improvement with antioxidant vitamin therapy. J Pediatr 1998;133:35-40.

62. Joannides R, Haefeli W E, Linder L et al. Nitric oxide is responsible for flow-dependent dilatation of human peripheral conduit arteries in vivo. Circulation 1995;91:1314-9.

63. Anderson T J, Uehata A, Gerhard M D et al. Close relation of endothelial function in the human coronary and peripheral circulations. J Am Coll Cardiol 1995;26: 1235-41.

64. Neunteufl T, Katzenschlager R, Hassan A et al. Systemic endothelial dysfunction is related to the extent and severity of coronary artery disease. Atherosclerosis 1997; 129:111-8.

65. Celermajer D S, Sorensen K E, Georgakopoulos D et al. Cigarette smoking is associated with dose-related and potentially reversible impairment of endothelium-dependent dilation in healthy young adults. Circulation 1993;88:2149-55.

66. Raitakari O T, Adams M R, McCredie R J, Griffiths K A, Celermajer D S. Arterial endothelial dysfunction related to passive smoking is potentially reversible in healthy young adults. Ann Intern Med 1999;130:578-81.

67. Karvonen M K, Pesonen U, Koulu M et al. Association of a leucine(7)-to-proline(7) polymorphism in the signal peptide of neuropeptide Y with high serum cholesterol and LDL cholesterol levels. Nat Med 1998;4:1434-7.

68. Niskanen L, Rauramaa R, Miettinen H, Haffner S M, Mercuri M, Uusitupa M. Carotid artery intima-media thickness in elderly patients with NIDDM and in nondiabetic subjects. Stroke 1996;27:1986-92.

1. A method for enhancing the endothelial function in humans, comprising administering to the person an NPY receptor active agent, wherein said receptor is present in the endothelial tissue.

2. The method according to claim 1 wherein said agent is an NPY receptor agonist.

3. The method according to claim 2 wherein said receptor is the Y2 receptor.

4. The method according to claim 3 wherein i) said agent also is a Y1-receptor agonist or antagonist, or ii) wherein a separate Y1 receptor agonist or antagonist is administered in combination with the Y2 receptor agonist.

5. A method for the treatment or prevention of atherosclerotic vascular diseases, comprising administering to the person an NPY receptor active agent, wherein said receptor is present in the endothelial tissue.

6. The method according to claim 5 wherein said agent is an NPY receptor agonist.

7. The method according to claim 6 wherein said receptor is the Y2 receptor.

8. The method according to claim 7 wherein i) said agent also is a Y1-receptor agonist or antagonist, or ii) wherein a separate Y1 receptor agonist or antagonist is administered in combination with the Y2 receptor agonist.

9. A method for the treatment or prevention of vascular spasm associated with angina pectoris, comprising administering to the person an NPY receptor active agent, wherein said receptor is present in the endothelial tissue.

10. The method according to claim 9 wherein said agent is an NPY receptor agonist.

11. The method according to claim 10 wherein said receptor is the Y2 receptor.

12. The method according to claim 11 wherein i) said agent also is a Y1-receptor agonist or antagonist, or ii) wherein a separate Y1 receptor agonist or antagonist is administered in combination with the Y2 receptor agonist.

13. A method for the treatment or prevention of micro- or macrovascular complications of diabetes, comprising administering to the person an NPY receptor active agent, wherein said receptor is present in the endothelial tissue.

14. The method according to claim 13 wherein said agent is an NPY receptor agonist.

15. The method according to claim 14 wherein said receptor is the Y2 receptor.

16. The method according to claim 15 wherein i) said agent also is a Y1-receptor agonist or antagonist, or ii) wherein a separate Y1 receptor agonist or antagonist is administered in combination with the Y2 receptor agonist.

17. A method for the treatment or prevention of any disease or disorder where a deficit in the formation of nitric oxide for the vascular endothelium appears evident, said method comprising administering to the person an NPY receptor active agent, wherein said receptor is present in the endothelial tissue.

18. The method according to claim 17 wherein said agent is an NPY receptor agonist.

19. The method according to claim 18 wherein said receptor is the Y2 receptor.

20. The method according to claim 19 wherein i) said agent also is a Y1-receptor agonist or antagonist, or ii) wherein a separate Y1 receptor agonist or antagonist is administered in combination with the Y2 receptor agonist.

21. A method for the treatment or prevention of premature ejaculation and impotence, said method comprising administering to the person an NPY receptor active agent, wherein said receptor is present in the endothelial tissue.

22. The method according to claim 21 wherein said agent is an NPY receptor agonist.

23. The method according to claim 22 wherein said receptor is the Y2 receptor.

24. The method according to claim 23 wherein i) said agent also is a Y1-receptor agonist or antagonist, or ii) wherein a separate Y1 receptor agonist or antagonist is administered in combination with the Y2 receptor agonist.
